SEQUENCE LISTING (1 sequence)

SEQ ID NO: 1 - protein, 195 aa, Homo sapiens
MALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

(19) United States
(12) Patent Application Publication
(10) Pub. No.: US 2026/0248890 A1
(43) Pub. Date: Aug. 27, 2026

(54) COMPOSITIONS AND METHODS FOR TREATING CANCER WITH INTERFERON-OMEGA AND ANALOGS THEREOF

(71) Applicants: Whitehead Institute for Biomedical Research, Cambridge, MA (US); Dana-Farber Cancer Institute, Inc., Boston, MA (US)

(72) Inventors: Kipp Weiskopf, Wellesley, MA (US); Asaf Maoz, Chestnut Hill, MA (US)

(73) Assignees: Whitehead Institute for Biomedical Research, Cambridge, MA (US); Dana-Farber Cancer Institute, Inc., Boston, MA (US)

(21) Appl. No.: 18/994,367

(22) PCT Filed: Jul. 14, 2023
(86) PCT No.: PCT/US2023/027792

§ 371 Date: Jan. 14, 2025

Related U.S. Application Data
(60) Provisional application No. 63/389,637, filed on Jul. 15, 2022.

(51) Int. Cl. A61K 38/21; A61K 47/68; A61P 35/00; C07K 14/555; C07K 16/28; G01N 33/50

(52) U.S. Cl. CPC A61K 38/21; A61K 47/6813; A61K 47/6851; A61P 35/00; C07K 14/555; C07K 16/2803; G01N 33/5011; G01N 33/5055; C07K 2319/30; G01N 2333/555

(57) ABSTRACT

The present disclosure relates to the discovery that IFN-ω activates macrophages to target cancer cells and can therefore be used as a therapeutic strategy for increasing macrophage-mediated cancer cytotoxicity in a subject. In one aspect, the present disclosure provides methods of treating a proliferative disease (e.g., cancer) in a subject in need thereof comprising administering to the subject a therapeutically effective amount of interferon-omega (IFN-ω) or an analog thereof, or an agent that increases endogenous interferon-omega (IFN-ω) expression in the subject.

### RELATED APPLICATIONS

This application claims priority under 35 U.S.C. § 119(e) to United States Provisional Patent Application, U.S. Ser. No. 63/389,637, filed Jul. 15, 2022, the entire contents of which is incorporated herein by reference.

### BACKGROUND

Therapies that activate macrophages are emerging in cancer immunotherapy. Macrophages are a component of many solid cancers and in some instances promote tumorigenesis by facilitating angiogenesis, immunosuppression, invasion, and metastasis. Given the association between high macrophage infiltration and poor survival in cancers, these immune cells represent promising targets for anticancer therapy. However, current therapeutic strategies focus on engaging immune cells other than macrophages (e.g., T-cells) to target cancer cells.

### SUMMARY

The present disclosure relates to the discovery that interferon-omega (IFN-ω) is useful in treating cancer. In particular, the present disclosure relates to experimental data showing that IFN-ω can promote macrophage-mediated cytotoxicity, e.g., by activating macrophages against cancer cells. These findings indicate that IFN-ω, and analogs thereof, can be used as a therapeutic strategy for treating proliferative diseases (e.g., cancer) by engaging macrophages. These findings also indicate that agents capable of increasing the expression of endogenous IFN-ω are also attractive therapeutic strategies for treating proliferative diseases (e.g., cancer).

The present disclosure also relates to the discovery that IFN-ω induces cancer cell death by macroptosis rather than phagocytosis, e.g., in a manner that depends on contact between the macrophages and the cancer cells. Therefore also provided herein are methods of identifying agents that induce cancer cell death by macroptosis over phagocytosis. The methods provided herein can be used to screen any candidate agents, including analogs of IFN-ω and agents comprising IFN-ω or analogs thereof.

In one aspect, provided herein are methods of treating a proliferative disease (e.g., cancer) in a subject in need thereof comprising administering to the subject a therapeutically effective amount of interferon-omega (IFN-ω) or an analog thereof. In certain embodiments, the IFN-ω or analog thereof is administered in an amount effective to increase macrophage-mediated cytotoxicity in the subject, relative to control. In certain embodiments, the macrophage-mediated cytotoxicity is increased by at least 10%, relative to control. In certain embodiments, the IFN-ω or analog thereof is human wild-type IFN-ω.

In another aspect, provided herein are methods of treating a proliferative disease (e.g., cancer) in a subject in need thereof comprising administering to the subject a therapeutically effective amount of an agent that increases endogenous interferon-omega (IFN-ω) expression in the subject, relative to control. In certain embodiments, the endogenous interferon-omega (IFN-ω) expression is increased by at least 10%, relative to control. In certain embodiments, the agent that increases endogenous IFN-ω expression is administered in an amount effective to increase macrophage-mediated cytotoxicity in the subject, relative to control. In certain embodiments, the macrophage-mediated cytotoxicity is increased by at least 10%, relative to control.

In certain embodiments of any of the methods provided herein, the proliferative disease is cancer. In certain embodiments, the subject has a tumor (e.g., a macrophage-infiltrated tumor). In certain embodiments, the cancer is colorectal cancer (e.g., colon adenocarcinoma). In certain embodiments, the cancer is breast cancer.

In another aspect, also provided herein are methods comprising administering to a subject interferon-omega (IFN-ω), or an analog thereof, in an amount effective to increase macrophage-mediated cytotoxicity in the subject, relative to control. In certain embodiments, the macrophage-mediated cytotoxicity is increased by at least 10%, relative to control. In certain embodiments, the subject is diagnosed with cancer. In certain embodiments, the subject has a tumor (e.g., a macrophage-infiltrated tumor). In certain embodiments, the macrophages are tumor-associated macrophages (TAMs). In certain embodiments, the IFN-ω, or analog thereof, is human wild-type IFN-ω.

Also provided herein are methods comprising administering to a subject an agent that increases endogenous interferon-omega (IFN-ω) expression in the subject, relative to control, in an amount effective to increase macrophage-mediated cytotoxicity in the subject, relative to control. In certain embodiments, the macrophage-mediated cytotoxicity is increased by at least 10%, relative to control. In certain embodiments, the subject is diagnosed with cancer. In certain embodiments, the subject has a tumor (e.g., a macrophage-infiltrated tumor). In certain embodiments, the macrophages are tumor-associated macrophages (TAMs).

In certain embodiments of any one of the methods provided herein, the subject is a human.

Also provided herein are pharmaceutical compositions and kits comprising IFN-ω, analogs thereof, or other agents described herein. The pharmaceutical compositions and kits are useful in the methods described herein.

### BRIEF DESCRIPTION OF THE DRAWINGS

The accompanying drawings, which are incorporated in and constitute a part of this specification, illustrate several embodiments of the disclosure and together with the description, provide non-limiting examples of the disclosure.

FIG. 1 shows that IFN-alpha, -gamma, and -omega activate macrophages to inhibit DLD1 cells (without CD47 blockade). Primary human macrophages were co-cultured in 384-well plates with GFP+ DLD-1 cancer cells. Macrophages and cells were co-cultured in the presence of interferon alpha 2A (IFNA2A), interferon alpha 2B (IFNA2B), interferon omega (IFNW1), and interferon gamma (IFNG), all at a final concentration 1000 ng/ml, or PBS control. GFP+ area was quantified at 7 days by automated microscopy and image analysis (Incucyte®). Cell numbers per well: Human macrophages: 10,000 (4 donors, each in triplicate); DLD-1: 7500.

FIG. 2 shows IFN-ω can activate macrophages against cell lines with mesenchymal properties (without CD47 blockade). Primary human macrophages were co-cultured in 384-well plates with GFP+ HMLER cancer cells with knockout of the EED or KMT2D genes (described in Zhang, Y., Donaher, J. L., Das, S. et al., “Genome-wide CRISPR screen identifies PRC2 and KMT2D-COMPASS as regulators of distinct EMT trajectories that contribute differentially to metastasis.” Nat Cell Biol. 2022, 24, 554-564). Macrophages and cells were co-cultured in the presence of interferon omega (IFNW1) at a final concentration 1000 ng/ml, or PBS control. GFP+ area was quantified at 7 days by automated microscopy and image analysis (Incucyte®). Cell numbers per well: Human macrophages: 10,000 (3 donors, each in triplicate); HMLER: 7500.

FIG. 3 shows human IFN-ω can activate murine macrophages against murine MC38 cells (without CD47 blockade). Murine C57BL/6 and NOD.Cg-Prkdc^{scid} Il2rg^{tm1wjl}/SzJ (NSG) macrophages were co-cultured in 384-well plates with GFP+ MC38 cancer cells. Macrophages and cells were co-cultured in the presence of interferon omega (IFNW1), interferon gamma (IFNG) or PBS control. Cells were co-cultured for up to 7 days and GFP+ area was quantified by automated microscopy and image analysis (Incucyte®). Each condition was plated in triplicate. Cell numbers per well: Murine macrophages: 10,000; MC38: 1000.

FIGS. 4A-4C demonstrate how a high-throughput functional screen identified IFN-ω as a cytokine that induces macrophage-dependent cell death of cancer cells.

FIGS. 5A-5B show that IFN-ω induces macroptosis of other human and mouse cell lines.

FIGS. 6A-6D show that IFN-ω induces cancer cell death by macroptosis, a non-phagocytic, contact-dependent form of macrophage-induced cell death.

FIGS. 7A-7D show that IFN-ω stimulates anti-tumor responses by myeloid cells in vivo.

FIG. 8 shows a CRISPR screen identifying genes that are required for sensitivity and resistance to macroptosis.

FIGS. 9A-9C show an IFN-ω-Fc fusion protein that binds to cancer cells and stimulates macroptosis.

### DETAILED DESCRIPTION

Described herein are compositions and methods for treating proliferative diseases (e.g., cancer) in a subject by administering interferon-omega (IFN-ω) or an analog thereof, or an agent that increases endogenous IFN-ω expression in the subject. In certain embodiments, the IFN-ω, analog thereof, or agent that increases endogenous IFN-ω expression is administered in an amount effective to increase macrophage-mediated cytotoxicity in the subject, relative to control. As described herein, the IFN-ω, analog thereof, or agent that increases endogenous IFN-ω expression may activate macrophages (e.g., tumor-associated macrophages (TAMs)) against cancer cells. Also provided herein are methods of screening candidate agents that induce cancer cell death by macroptosis over phagocytosis.

### IFN-ω, Analogs Thereof, and Agents

Interferon-ω (IFN-ω; IFNW1) is a protein that in humans is encoded by the IFNW1 gene. The sequence of human wild-type interferon-omega (IFN-ω) is provided below as SEQ ID NO: 1.

(SEQ ID NO: 1)
MALLFPLLAALVMTSYSPVGSLGCDLPQNHGLLSRNTLVLLHQMRRISPFLCLKDRRDFR
FPQEMVKGSQLQKAHVMSVLHEMLQQIFSLFHTERSSAAWNMTLLDQLHTGLHQQLQHLE
TCLLQVVGEGESAGAISSPALTLRRYFQGIRVYLKEKKYSDCAWEVVRMEIMKSLFLSTN
MQERLRSKDRDLGSS

In certain embodiments of the methods provided herein, the IFN-ω is a wild-type IFN-ω. In certain embodiments of the methods provided herein, the IFN-ω is human wild-type IFN-ω. In certain embodiments of the methods provided herein, the IFN-ω is non-human IFN-ω. In certain embodiments, the IFN-ω is feline IFN-ω (e.g., Virbagen® Omega). In certain embodiments of the methods provided herein, an analog of IFN-ω is used. The IFN-ω analog may be naturally occurring, recombinant, synthetic, modified, or any combination of these.

As used herein, the term “analog” refers to a protein of similar amino acid composition or sequence to any of the proteins or peptides of the disclosure (e.g., IFN-ω), allowing for variations that do not have an adverse effect on the ability of the protein or peptide to carry out its normal function (e.g., bind to a ligand, initiate downstream signaling, and/or stimulate or activate macrophages). Analogs may be the same length, shorter, or longer than their corresponding protein or polypeptide. Analogs include homologs, variants (including muteins), and fragments of their corresponding protein or polypeptide. Analogs may have at least about 60% (e.g., at least about 60%, at least about 62%, at least about 64%, at least about 66%, at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 98%, or at least about 99%) identity to the amino acid sequence of the protein or peptide. An analog can be a naturally occurring protein or polypeptide sequence that is modified by deletion, addition, mutation, or substitution of one or more amino acid residues. In certain embodiments, the analog is a fragment of the corresponding protein.

For example, an IFN-ω analog may have one or more amino acid deletions, additions, mutations, or substitutions relative to naturally occurring IFN-ω, e.g., SEQ ID NO: 1. In certain embodiments, an IFN-ω analog has 1-20 mutations, inclusive, relative to naturally occurring IFN-ω, e.g., SEQ ID NO: 1. In certain embodiments, an IFN-ω analog has 1-10 mutations, inclusive, relative to naturally occurring IFN-ω, e.g., SEQ ID NO: 1. In certain embodiments, an IFN-ω analog has 1-5 mutations, inclusive, relative to naturally occurring IFN-ω, e.g., SEQ ID NO: 1. In certain embodiments, an IFN-ω analog has 1 mutation relative to naturally occurring IFN-ω, e.g., SEQ ID NO: 1. In certain embodiments, an IFN-ω analog has 2 mutations relative to naturally occurring IFN-ω, e.g., SEQ ID NO: 1. In certain embodiments, an IFN-ω analog has 3 mutations relative to naturally occurring IFN-ω, e.g., SEQ ID NO: 1. In certain embodiments, an IFN-ω analog has 4 mutations relative to naturally occurring IFN-ω, e.g., SEQ ID NO: 1. In certain embodiments, an IFN-ω analog has 5 mutations relative to naturally occurring IFN-ω, e.g., SEQ ID NO: 1. In certain embodiments, an IFN-ω analog has 6 mutations relative to naturally occurring IFN-ω, e.g., SEQ ID NO: 1. In certain embodiments, an IFN-ω analog has 7 mutations relative to naturally occurring IFN-ω, e.g., SEQ ID NO: 1. In certain embodiments, an IFN-ω analog has 8 mutations relative to naturally occurring IFN-ω, e.g., SEQ ID NO: 1. In certain embodiments, an IFN-ω analog has 9 mutations relative to naturally occurring IFN-ω, e.g., SEQ ID NO: 1. In certain embodiments, an IFN-ω analog has 10 mutations relative to naturally occurring IFN-ω, e.g., SEQ ID NO: 1.

In certain embodiments, the IFN-ω analog has at least about 60% sequence identity to SEQ ID NO: 1. In certain embodiments, the IFN-ω analog has at least about 65% sequence identity to SEQ ID NO: 1. In certain embodiments, the IFN-ω analog has at least about 70% sequence identity to SEQ ID NO: 1. In certain embodiments, the IFN-ω analog has at least about 75% sequence identity to SEQ ID NO: 1. In certain embodiments, the IFN-ω analog has at least about 80% sequence identity to SEQ ID NO: 1. In certain embodiments, the IFN-ω analog has at least about 85% sequence identity to SEQ ID NO: 1. In certain embodiments, the IFN-ω analog has at least about 90% sequence identity to SEQ ID NO: 1. In certain embodiments, the IFN-ω analog has at least about 95% sequence identity to SEQ ID NO: 1. In certain embodiments, the IFN-ω analog has at least about 98% sequence identity to SEQ ID NO: 1. In certain embodiments, the IFN-ω analog has at least about 99% sequence identity to SEQ ID NO: 1.

In certain embodiments, the IFN-ω or analog thereof is modified in order to enhance one or more of its properties (e.g., pharmacokinetic parameters, efficacy, immunogenicity). In certain embodiments, the IFN-ω or analog thereof is modified chemically, i.e., a chemical moiety is attached to the IFN-ω or analog thereof by formation of new chemical bond(s). In certain embodiments, the IFN-ω or analog thereof is modified by, for example, pegylation, glycosylation, albumin (e.g., human serum albumin (HSA)) conjugation, or hesylation. For example, in certain embodiments, a modification (e.g., pegylation) can increase in vivo half-life of the IFN-ω or analog thereof. In some embodiments, modification of the IFN-ω or analog thereof does not result in a therapeutically relevant, detrimental effect on immunogenicity, and in some embodiments the modified IFN-ω or analog thereof is less immunogenic than unmodified the IFN-ω or analog thereof. In certain embodiments, the IFN-ω or analog thereof is pegylated.

In certain embodiments of the methods provided herein, an agent that increases endogenous IFN-ω expression in a subject is used. In certain embodiments, the agent increases endogenous IFN-ω expression in a subject by at least about 10% (e.g., at least about 20%, at least about 30%, at least about 40%, at least about 50%, at least about 60%, at least about 70%, at least about 80%, at least about 90%, at least about 95%, at least about 98%, at least about 99%), relative to control.

In certain embodiments, the agent that increases endogenous IFN-ω expression is a small molecule, protein, polypeptide, antibody or antigen-binding fragment thereof, or nucleic acid. In certain embodiments, the agent is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present disclosure.

“Small molecules” include organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, e.g., organic or inorganic compounds having a molecular weight less than about 250 grams per mole, e.g., organic or inorganic compounds having a molecular weight less than about 100 grams per mole and salts, esters, and other pharmaceutically acceptable forms of such compounds.

The terms “peptide” and “polypeptide” are used interchangeably and refer to a polymer of amino acid residues linked together by peptide bonds. The terms also include “proteins,” and refer to peptides, polypeptides, and proteins, of any size, structure, or function. Typically, a peptide will be at least three amino acids long, or at least the length required by an amino acid sequence provided herein. A peptide may refer to an individual peptide or a collection of peptides. Peptides provided herein can include natural amino acids and/or unnatural amino acids (i.e., compounds that do not occur in nature but that can be incorporated into a peptide chain) in any combination. A peptide may be a fragment or modified version of a naturally occurring peptide or protein. A peptide may be naturally occurring, recombinant, synthetic, or any combination of these.

“Antibody” refers to a molecule that specifically binds to, or is immunologically reactive with, a particular antigen and includes at least the variable domain of a heavy chain, and normally includes at least the variable domains of a heavy chain and of a light chain of an immunoglobulin. Unless otherwise indicated, the term “antibody” (Ab) is meant to include both intact (whole) molecules as well as antibody fragments (e.g., Fab and F(ab′)₂ fragments) that are capable of specifically binding to a target antigen. Antibodies (including intact antibodies and antigen-binding fragments), variants, and derivatives thereof include, but are not limited to, polyclonal, monoclonal, multispecific, human, humanized, primatized, or chimeric antibodies, heteroconjugate antibodies (e.g., bi- tri- and quad-specific antibodies, diabodies, triabodies, and tetrabodies), single-domain antibodies (sdAb), epitope-binding fragments (e.g., Fab, Fab′, and F(ab′)₂, Fd, Fvs, single-chain Fvs (scFv), rlgG, single-chain antibodies, disulfide-linked Fvs (sdFv), fragments containing either a V_{L} or V_{H} domain, fragments produced by an Fab expression library), and anti-idiotypic (anti-Id) antibodies. Fab and F(ab′)₂ fragments, for example, lack the Fc fragment of an intact antibody. Antibody molecules of the conjugates can be of any type (e.g., IgG, IgE, IgM, IgD, IgA, and IgY), class (e.g., IgG1, IgG2, IgG3, IgG4, IgA1 and IgA2) or subclass of immunoglobulin molecule. Antibodies described herein can be murine, rat, human, or of any other origin (including chimeric or humanized antibodies and fragments thereof).

“Antigen-binding fragment,” as used herein, refers to one or more fragments of an immunoglobulin that retain the ability to specifically bind to a target antigen. The antigen-binding function of an immunoglobulin can be performed by fragments of a full-length antibody. The antibody fragments can be, e.g., a Fab, F(ab′)₂, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment containing two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb (Ward et al., Nature, 1989, 341, 544-546) including V_{H} and V_{L} domains; (vi) a dAb fragment that consists of a V_{H} domain; (vii) a dAb that consists of a V_{H} or a V_{L} domain; (viii) an isolated complementarity determining region (CDR); and (ix) a combination of two or more isolated CDRs which may optionally be joined by a linker, e.g., a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv)). These antibody fragments (i.e., “antigen-binding fragments”) can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

“Nucleic acid”, “polynucleotide”, and “oligonucleotide” are used interchangeably and refer to a series of nucleotide bases (also called “nucleotides”) in DNA and RNA, and mean any chain of two or more nucleotides. The nucleic acids can be chimeric mixtures or derivatives or modified versions thereof, single-stranded or double-stranded. The nucleic acid can be modified at the base moiety, sugar moiety, or phosphate backbone, for example, to improve stability of the molecule, its hybridization parameters, etc. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

In certain embodiments, the IFN-ω, analog thereof, or agent that increases expression of IFN-ω is part of a fusion protein (e.g., Fc fusion protein) or other bispecific agent. In certain embodiments, the IFN-ω, analog thereof, or agent that increases expression of IFN-ω is part of a fusion protein (e.g., Fc fusion protein). In certain embodiments, the fusion protein is an Fc fusion protein. In certain embodiments, the IFN-ω, analog thereof, or agent that increases expression of IFN-ω is conjugated to an antibody or antigen-binding fragment thereof (“antigen-binding domain”). For example, the IFN-ω, analog thereof, or agent that increases expression of IFN-ω can be conjugated to an anti-cancer antibody, e.g., for targeted delivery to a cancer or tumor microenvironment. For example, the IFN-ω, analog thereof, or agent that increases expression of IFN-ω can be conjugated to an anti-CD20-, anti-EGFR, or anti-HER2 antibody or antigen-binding fragment thereof. For example, the IFN-ω, analog thereof, or agent that increases expression of IFN-ω can be conjugated to an anti-cancer antibody such as rituximab, cetuximab, trastuzumab. Other non-limiting examples of anti-cancer antibodies are provided herein.

As described, provided herein are fusion proteins comprising IFN-ω, or an analog thereof, and an antigen-binding domain. For example, the antigen-binding domain can be an antibody, antibody-binding fragment thereof, a nanobody, or other protein. In some embodiments the fusion protein can comprise IFN-ω or an analog thereof, an antigen binding domain, and a half-life extending domain such as albumin or an XTEN polypeptide.

In some aspects, provided herein are fusion proteins comprising IFN-ω or an analog thereof, an Fc domain, and an antigen-binding domain (“IFN-ω-Fc fusion proteins”). In some embodiments, the antigen-binding domain is a CD47-binding domain. In some embodiments, the CD47-binding domain is CV1 or an analog thereof (see Weiskopf et al., Science 2013, 341, 88-91; the entire contents of which are hereby incorporated by reference). In certain embodiments, the CD47-binding domain is CV1.

In some embodiments, the IFN-ω-Fc fusion protein comprises a mammalian Fc domain. In some embodiments, the IFN-ω-Fc fusion protein comprises a human Fc domain. In some embodiments, the human Fc domain is a human IgG1, human IgG2, human IgG3, human IgA1, human IgA2, human IgE, human IgD, or human IgM domain. In some embodiments, the human Fc domain is a human IgG4 Fc. In some embodiments, the Fc domain comprises one or more mutations that enhance effector function or increase binding to Fc receptors. In some embodiments, the Fc domain comprises one or more mutations that decrease effector function or binding to Fc receptors. In some embodiments, the Fc domain is silent human IgG1 comprising mutations that decrease effector functions (e.g., PG-LALA mutations). In some embodiments, the Fc domain is human IgG4 Fc comprising an S228P mutation.

In some embodiments of the IFN-ω-Fc fusion proteins, the antigen-binding domain is operably linked to the Fc domain, and the Fc domain is operably linked to the IFN-ω or analog thereof. In some embodiments, the IFN-ω or analog thereof is operably linked to the C-terminus of the Fc domain. In some embodiments, the IFN-ω or analog thereof is operably linked to the C-terminus of the Fc domain, and the Fc domain is operably linked to the C-terminus of the antigen-binding domain. “Operably linked” may be a direct linkage via a bond, or linkage via a linker.

In certain embodiments, a nucleic acid that encodes IFN-ω or an analog thereof is used in the methods described herein. In certain embodiment, the nucleic acid is an mRNA that encodes IFN-ω or an analog thereof.

In certain embodiments, stimulator of interferon genes protein (STING) agonist is used in the methods described herein.

Without wishing to be bound by any particular theory, IFN-ω and analogs thereof act by binding one or more IFNAR receptors. Therefore, provided herein are agents (e.g., IFN-ω and analogs thereof) that bind one or more IFNAR receptors to induce macroptosis. In another aspect, provided herein are agents (e.g., IFN-ω and analogs thereof) that act by binding one or more other IFN-ω receptors, e.g., to induce macroptosis. In certain embodiments, the agent is competitive with IFN-ω for binding or function. In certain embodiments, the agent is an analog of IFN-ω. In certain embodiments, the agent is an antibody, nanobody, recombinant protein, fusion protein, peptide, small molecule, or other agent.

### Macrophage-Mediated Cytotoxicity

As described herein, the present disclosure is based in part on the discovery that IFN-ω activates macrophages to kill cancer cells. In certain embodiments, the IFN-ω, analog thereof, or agent that increases expression of IFN-ω described herein is administered to a subject in an amount effective to increase macrophage-mediated cytotoxicity in the subject, relative to control. In certain embodiments, the IFN-ω, analog thereof, or agent that increases endogenous IFN-ω expression described herein is administered to a subject in an amount effective to increase macrophage-mediated cytotoxicity in the subject by at least 10%, e.g., at least about 20%, at least about 30%, at least about 40%, at least about 50%, at least about 60%, at least about 70%, at least about 80%, at least about 90%, at least about 95%, at least about 98%, at least about 99%), relative to control. In certain embodiments, the macrophages are tumor-associated macrophages (TAMs).

“Macrophage” refers to a type of white blood cell of the immune system (i.e., immune cell) that can engulf and digest pathogens, such as cancer cells, microbes, cellular debris, and foreign substances, which do not have proteins that are specific to healthy body cells on their surface. This process is called phagocytosis, which acts to defend the host against infection and injury. Types of macrophages include, but are not limited to, peripheral macrophages, tissue resident macrophages, and tumor-associated macrophages (TAMs), M1 macrophages, and M2 macrophages. “Tumor-associated macrophages” (TAMs) are macrophages that participate in the formation of the tumor microenvironment and/or are present in the tumor microenvironment. TAMs are widely present in various tumors and are traditionally known to promote tumor growth, invasion, metastasis, and drug resistance. See, e.g., Pen et al., “Tumor-Associated Macrophages in Tumor Immunity” Front. Immunol. 2020, 11, 1-9. As described herein, the present invention shows that TAMs can be activated to turn against (i.e., attack) cancer cells.

“Cytotoxicity,” as used herein, refers to the ability of immune cells to kill cancer cells. Natural killer cells, ILCs, and cytotoxic T-cells (e.g., CD8+ T-cells) are among the primary cytotoxic effector cells of the immune system, although dendritic cells, neutrophils, eosinophils, mast cells, basophils, macrophages, and monocytes have been shown to have cytotoxic activity. “Macrophage-mediated cytotoxicity” refers to the direct or indirect killing of cancer cells by macrophages. Macrophages, once activated, can kill cancer cells by themselves in a direct manner or indirectly through recruitment of other immune cells, such as cytotoxic T-lymphocytes. Possible mechanisms of macrophage-mediated cytotoxicity include, but are not limited to: (1) Direct killing through the release of harmful products (e.g., reactive oxygen species (ROS), tumor necrosis factor (TNF), nitric oxide (NO)); (2) direct phagocytosis; (3) direct cytolysis of cancer cells through antibody-dependent cellular cytotoxicity (ADCC); (4) indirect killing by recruitment of other immune cells that can lyse the cancer cells (e.g., cytotoxic T-cells); or any combination of the foregoing.

The terms “increase” or “increasing” in the context of expression or activity (e.g., of a biomolecule or biological function) refers to an increase in the level of expression or activity to a level that is reproducibly and/or statistically significantly higher than an initial or other appropriate reference level (i.e., control), which may, for example, be a baseline level of activity or expression. For example, an increase in expression of endogenous IFN-ω in a subject refers to an increase in the level of IFN-ω in the subject that is reproducibly and/or statistically significantly higher than an initial or other appropriate reference level (e.g., the level before administration of an agent described herein). As another example, an increase in macrophage-mediated cytotoxicity refers to an increase in the level of macrophage-mediated cytotoxicity that is reproducibly and/or statistically significantly higher than an initial or other appropriate reference level (e.g., the level before administration of IFN-ω, analog thereof, or agent that increases endogenous IFN-ω expression as described herein). In certain embodiments, an increase is by at least 10%, e.g., at least about 20%, at least about 30%, at least about 40%, at least about 50%, at least about 60%, at least about 70%, at least about 80%, at least about 90%, at least about 95%, at least about 98%, or at least about 99% of an initial level of expression or activity, which may, for example, be a baseline level of expression or activity. In certain embodiments, the increase is at least a 2-fold, at least a 3-fold, at least a 4-fold, at least a 5-fold, at least a 6-fold, at least a 7-fold, at least a 8-fold, at least a 9-fold, at least a 10-fold, at least a 20-fold, at least a 30-fold, at least a 40-fold, at least a 50-fold, at least a 60-fold, at least a 70-fold, at least a 80-fold, at least a 90-fold, at least a 100-fold, at least a 1000-fold, at least a 10000-fold, or at least a 100000-fold greater than an initial level of expression or activity, which may, for example, be a baseline level of expression or activity.

Without wishing to be bound by any particular theory, other immune cells may also contribute to the cytotoxic effects of the agents described herein. For example, monocytes, granulocytes, neutrophils, dendritic cells, or other immune cell types may contribute the cytotoxic effect (e.g., in vivo), in addition to or instead of the action of macrophages described herein.

Macrophage-Mediated Cytotoxicity by Macroptosis Rather than Phagocytosis

The present disclosure in part relates to the discovery that IFN-ω induces cancer cell death by macroptosis rather than phagocytosis, e.g., in a manner that is dependent on contact between the cancer cells the macrophages. As described herein, “macroptosis” is a non-phagocytic, contact-dependent form of macrophage-induced cell death.

In some embodiments, increased macroptosis is identified by observing an increase in cancer cell death in the presence of macrophages, relative to control, while observing the same or similar level of phagocytosis, relative to control.

In some embodiments, an increase in macroptosis is identified by observing an increase in cancer cell death when macrophages are able to directly contact the cancer cells relative to a control condition, while observing the same or similar level of cancer cell death relative to a control condition when the macrophages cannot directly contact the cancer cells. In some embodiments, transwell assays are used to identify macroptosis-mediated cell killing rather than phagocytosis-mediated cell killing. In some embodiments, (i) microscopy, (ii) flow cytometry, (iii) annexin V staining, (iv) caspase activation assays, (v) ELISA, (vi) an ATP assay, (vii) and/or a cell membrane integrity assay is used to identify macroptosis-mediated cell killing rather than phagocytosis-mediated cell killing.

In some embodiments, phagocytosis is measured by internalization of fluorescent marker-expressing cells. In some embodiments, phagocytosis is measured by co-culturing macrophages with GFP-expressing cancer cells and subsequently measuring fluorescence of the macrophages by flow cytometry. Without intending to be bound by theory, macrophages that internalize GFP-expressing cells will be counted by flow cytometry, so the number of fluorescent macrophages counted by flow cytometry indicates the level of phagocytosis by macrophages. In some embodiments, phagocytosis assays use human macrophages and DLD-1 cancer cells. In some embodiments, phagocytosis assays use C57BL/6 mouse macrophages and MC38 cancer cells.

For example, provided herein are methods of identifying an agent that induces cancer cell death by macroptosis over phagocytosis, the method comprising:
(i) co-culturing cancer cells and macrophages with a candidate agent, wherein the cancer cells and macrophages are capable of direct contact with each other;
(ii) detecting or measuring the total level or rate of cancer cell death;
(iii) detecting or measuring the level or rate of cancer cell death due to phagocytosis; and
(iv) selecting the candidate agent as an agent that induces cancer cell death by macroptosis over phagocytosis if the total level or rate of cancer cell death is increased by a statistically significant amount, relative to control, and the level or rate of cancer cell death due to phagocytosis is not increased by a statistically significant amount, relative to control.

In some embodiments, the macrophages are primary human macrophages or C57BL/6 mouse macrophages. In some embodiments, the cancer cells comprise GFP+ cancer cells and the level or rate of cancer cell death due to phagocytosis is measured by flow cytometry. In some embodiments, the cancer cells are obtained from a subject. In some embodiments, the cancer cells are DLD-1 or MC38 cancer cells.

### Pharmaceutical Compositions, Kits, and Administration

The present disclosure provides pharmaceutical compositions comprising IFN-ω, an analog thereof, or agent that increases expression of IFN-ω described herein (i.e., the “active ingredient”). The pharmaceutical composition may comprise one or more pharmaceutically acceptable excipients or carriers. In certain embodiments, the active ingredient described herein is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include bringing the active ingredient into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, Eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle. Adjuvants such as aluminum may also be used.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an active ingredient described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the active ingredient in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Formulations for topical administration may further comprise one or more of the additional ingredients described herein.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions can be conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and are conveniently provided in a unit dose form. Low-boiling propellants generally include liquid propellants having a boiling point of below 65° F. at atmospheric pressure. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

A pharmaceutical composition described herein can be prepared, packaged, and/or sold in a formulation for buccal administration. Such formulations may, for example, be in the form of tablets and/or lozenges made using conventional methods. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Active ingredients provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The active ingredients and compositions provided herein can be administered by any route, including enteral (e.g., oral), parenteral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically, contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

In certain embodiments, the active ingredient or composition is administered locally (e.g., to the site of a tumor).

The exact amount of an active ingredient required to achieve an effective amount will vary from subject to subject, depending, for example, on species, age, and general condition of a subject, severity of the side effects or disorder, identity of the particular active ingredient, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an active ingredient described herein.

An active ingredient or composition, as described herein, can be administered in combination with one or more additional pharmaceutical agents (e.g., therapeutically and/or prophylactically active agents). The active ingredients or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including an active ingredient described herein and an additional pharmaceutical agent shows a synergistic effect that is absent in a pharmaceutical composition including one of the active ingredient or the additional pharmaceutical agent, but not both. In some embodiments, the additional pharmaceutical agent achieves a desired effect for the same disorder. In some embodiments, the additional pharmaceutical agent achieves different effects.

The active ingredient or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

The additional pharmaceutical agents include, but are not limited to, anti-proliferative agents, anti-cancer agents, anti-angiogenesis agents, steroidal or non-steroidal anti-inflammatory agents (NSAIDs), immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, and hormones.

In certain embodiments, the additional pharmaceutical agent is an anti-proliferative agent. In certain embodiments, the additional pharmaceutical agent is an anti-cancer agent. “Anti-cancer agents” encompass biotherapeutic anti-cancer agents as well as chemotherapeutic agents.

Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g., Herceptin (trastuzumab), T-DM1, AVASTIN (bevacizumab), ERBITUX (cetuximab), Vectibix (panitumumab), Rituxan (rituximab), Bexxar (tositumomab)). Other examples of biotherapeutic anti-cancer agents include PD-1 and PD-L1 inhibitors including, but not limited to, pembrolizumab (Keytruda), nivolumab (Opdivo), cemiplimab (Libtayo), and dostarlimab (Jemperli).

Exemplary chemotherapeutic agents include, but are not limited to, anti-estrogens (e.g. tamoxifen, raloxifene, and megestrol), LHRH agonists (e.g., goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (Abraxane), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA@), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In certain embodiments, the additional therapeutic agent is an immunotherapy. In certain embodiments, the additional therapeutic agent is a checkpoint inhibitor.

Each additional pharmaceutical agent may be administered at a dose and/or on a time schedule determined for that pharmaceutical agent. The additional pharmaceutical agents may also be administered together with each other and/or with the active ingredient or composition described herein in a single dose or composition or administered separately in different doses or compositions. The particular combination to employ in a regimen will take into account compatibility of the active ingredient described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the active ingredients described herein or pharmaceutical compositions can be administered in combination with an anti-cancer therapy including, but not limited to, surgery, radiation therapy, and transplantation (e.g., stem cell transplantation, bone marrow transplantation).

Also encompassed by the disclosure are kits (e.g., pharmaceutical packs). The kits provided may comprise a pharmaceutical composition or active ingredient described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or active ingredient described herein. In some embodiments, the pharmaceutical composition or active ingredient described herein provided in the first container and the second container are combined to form a single unit dosage form. Thus, in one aspect, provided are kits including a first container comprising a active ingredient or pharmaceutical composition described herein. In certain embodiments, the kits are useful for treating a disease (e.g., cancer) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits provide instructions for treating a disease (e.g., cancer) in a subject in need thereof. In certain embodiments, the kits provide instructions for preventing a disease in a subject in need thereof. A kit described herein may include one or more additional pharmaceutical agents described herein as a separate composition.

### Methods of Treatment and Uses

As described herein, provided are methods of treating and/or preventing proliferative diseases (e.g., cancer) with IFN-ω, analog thereof, or agent that increases expression of IFN-ω described herein. As described, the IFN-ω, analog thereof, or agent that increases IFN-ω expression can activate macrophages and increase macrophage-mediated cytotoxicity. In certain embodiments, the methods are for treating a proliferative disease (e.g., cancer). In certain embodiments, the methods are for preventing a proliferative disease.

In one aspect, provided herein are methods of treating a proliferative disease in a subject in need thereof comprising administering to the subject a therapeutically effective amount of interferon-omega (IFN-ω) or an analog thereof. In certain embodiments, the IFN-ω or analog thereof is administered in an amount effective to increase macrophage-mediated cytotoxicity in the subject, relative to control. In certain embodiments, the proliferative disease is cancer. Methods of treating cancer provided herein include methods of inhibiting tumor growth in a subject.

In another aspect, provided herein are method of treating a proliferative disease in a subject in need thereof comprising administering to the subject a therapeutically effective amount of an agent that increases endogenous interferon-omega (IFN-ω) expression in the subject, relative to control. In certain embodiments, the agent that increases endogenous IFN-ω expression is administered in an amount effective to increase macrophage-mediated cytotoxicity in the subject, relative to control. In certain embodiments, the proliferative disease is cancer. Methods of treating cancer provided herein include methods of inhibiting tumor growth in a subject.

In some embodiments, the IFN-ω, analog thereof, or agent that increases expression of IFN-ω described herein is administered to a subject in an amount effective to increase macrophage-mediated cytotoxicity by macroptosis in the subject, relative to a control.

As described herein, in certain embodiments, the proliferative disease is a cancer or tumor. In certain embodiments, the subject has a tumor. In certain embodiments, the tumor is a macrophage-infiltrated tumor. “Macrophage-infiltrated tumor” refers to a tumor containing tumor-associated macrophages (TAMs).

In certain embodiments the cancer is colorectal cancer (e.g., colon adenocarcinoma). In certain embodiments, the cancer is breast cancer.

Also provided herein are methods comprising administering to a subject interferon-omega (IFN-ω) or an analog thereof in an amount effective to increase macrophage-mediated cytotoxicity in the subject, relative to control. Additionally, provided herein are methods comprising administering to a subject an agent that increases endogenous interferon-omega (IFN-ω) expression in the subject, relative to control, in an amount effective to increase macrophage-mediated cytotoxicity in the subject, relative to control.

In certain embodiments, the subject is diagnosed with a proliferative disease. In certain embodiments, the subject is diagnosed with a cancer or tumor. In certain embodiments, the subject has a tumor. In certain embodiments, the tumor is a macrophage-infiltrated tumor.

In certain embodiments the cancer is colorectal cancer (e.g., colon adenocarcinoma). In certain embodiments, the cancer is breast cancer.

As shown in FIG. 1, IFN-α (e.g., IFNa1 or IFNa2) can also induce cancer cell death my macroptosis. Provided herein are methods of treating and/or preventing proliferative diseases (e.g., cancer) with IFN-α (e.g., IFNa1 or IFNa2), an analog thereof, or agent that increases expression of IFN-α. As described, the IFN-α (e.g., IFNa1 or IFNa2), analog thereof, or agent that increases expression of IFN-α can activate macrophages and increase macrophage-mediated cytotoxicity. In certain embodiments, the methods are for treating a proliferative disease (e.g., cancer). In certain embodiments, the methods are for preventing a proliferative disease.

In one aspect, provided herein are methods of treating a proliferative disease in a subject in need thereof comprising administering to the subject a therapeutically effective amount of IFN-α (e.g., IFNa1 or IFNa2) or an analog thereof. In certain embodiments, the IFN-α (e.g., IFNa1 or IFNa2) or an analog thereof is administered in an amount effective to increase macrophage-mediated cytotoxicity in the subject, relative to control. In certain embodiments, the proliferative disease is cancer. Methods of treating cancer provided herein include methods of inhibiting tumor growth in a subject.

In another aspect, provided herein are method of treating a proliferative disease in a subject in need thereof comprising administering to the subject a therapeutically effective amount of an agent that increases endogenous IFN-α (e.g., IFNa1 or IFNa2) expression in the subject, relative to control. In certain embodiments, the agent that increases endogenous IFN-α (e.g., IFNa1 or IFNa2) expression is administered in an amount effective to increase macrophage-mediated cytotoxicity in the subject, relative to control. In certain embodiments, the proliferative disease is cancer. Methods of treating cancer provided herein include methods of inhibiting tumor growth in a subject.

In some embodiments, the IFN-α (e.g., IFNa1 or IFNa2), analog thereof, or agent that increases expression of IFN-α described herein is administered to a subject in an amount effective to increase macrophage-mediated cytotoxicity by macroptosis in the subject, relative to a control.

As described herein, in certain embodiments, the proliferative disease is a cancer or tumor. In certain embodiments, the subject has a tumor. In certain embodiments, the tumor is a macrophage-infiltrated tumor. “Macrophage-infiltrated tumor” refers to a tumor containing tumor-associated macrophages (TAMs).

In certain embodiments the cancer is colorectal cancer (e.g., colon adenocarcinoma). In certain embodiments, the cancer is breast cancer.

Also provided herein are methods comprising administering to a subject IFN-α (e.g., IFNa1 or IFNa2), or analog thereof, in an amount effective to increase macrophage-mediated cytotoxicity in the subject, relative to control. Additionally, provided herein are methods comprising administering to a subject an agent that increases endogenous IFN-α (e.g., IFNa1 or IFNa2) expression, relative to control, in an amount effective to increase macrophage-mediated cytotoxicity in the subject, relative to control.

In certain embodiments, the subject is diagnosed with a proliferative disease. In certain embodiments, the subject is diagnosed with a cancer or tumor. In certain embodiments, the subject has a tumor. In certain embodiments, the tumor is a macrophage-infiltrated tumor.

In certain embodiments the cancer is colorectal cancer (e.g., colon adenocarcinoma). In certain embodiments, the cancer is breast cancer.

A “proliferative disease” refers to a disease that occurs due to abnormal growth or extension by the multiplication of cells (See, e.g., Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located.

The term “cancer” refers to a class of diseases characterized by the development of abnormal cells that proliferate uncontrollably and have the ability to infiltrate and destroy normal body tissues. In certain embodiments, the cancer is a solid tumor. In certain embodiments, the cancer is a hematopoietic cancer (i.e., hematological cancer).

In certain embodiments, the cancer is a hematopoietic cancer (e.g., leukemia (e.g., acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)); lymphoma (e.g., Hodgkin lymphoma (HL) (e.g., B-cell HL, T-cell HL)), non-Hodgkin lymphoma (NHL) (e.g., B-cell NHL such as diffuse large cell lymphoma (DLCL) (e.g., diffuse large B-cell lymphoma)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomads, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstram's macroglobulinemia), hairy cell leukemia (HCL), immunoblastic large cell lymphoma, precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome)), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); heavy chain disease (e.g., alpha chain disease, gamma chain disease, mu chain disease); a myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); multiple myeloma (MM); plasma cell neoplasia; familiar hypereosinophilia; inflammatory myofibroblastic tumors; immunocytic amyloidosis). In certain embodiments, the cancer is leukemia. In certain embodiments, the cancer is acute lymphoblastic leukemia (ALL). In certain embodiments, the cancer is early T-cell precursor (ETP)-acute lymphoblastic leukemia (ALL).

In certain embodiments, the cancer is liver cancer (e.g., hepatocellular cancer (HCC) (e.g., hepatocellular carcinoma, hepatoblastoma, hepatocellular adenoma), malignant hepatoma, hemangiomas, biliary cancer (e.g., cholangiocarcinoma)).

In certain embodiments, the cancer is musculoskeletal cancer (e.g., bone cancer (e.g., osteosarcoma, osteoid osteoma, malignant fibrous histiocytoma, Ewing's sarcoma, chordoma, malignant giant cell tumor chordoma, chondrosarcoma osteochondroma, benign chondroma, chondroblastoma chondromyxofibroma, myelodysplastic syndrome (MDS)), muscle cancer (e.g., rhabdomyosarcoma, rhabdomyoma), connective tissue cancer, synovioma).

In certain embodiments, the cancer is a nervous system cancer (e.g., brain cancer (e.g., astrocytoma, medulloblastoma, glioma (e.g., astrocytoma, oligodendroglioma), glioblastomas, glioblastoma multiform, medulloblastoma, ependymoma, germinoma (i.e., pinealoma), oligodendroglioma, schwannoma, retinoblastoma, congenital tumors, craniopharyngioma), spinal cord cancer, neurofibroma (e.g., neurofibromatosis (NF) type 1 or type 2, schwannomatosis), neuroblastoma, primitive neuroectodermal tumors (PNT), meningeal cancer (e.g., meningioma, meningiosarcoma, gliomatosis), skull cancer, acoustic neuroma, ependymoma, hemangioblastoma, ocular cancer (e.g., intraocular melanoma, retinoblastoma)). In certain embodiments, the disease to be treated is a brain tumor. In certain embodiments, the disease is pleomorphic xenoanthrocytoma (PXA). In certain embodiments, the disease is pediatric pleomorphic xenoanthrocytoma (PXA).

In certain embodiments, the cancer is selected from endocrine/exocrine cancers (e.g., thyroid cancer (e.g., papillary thyroid carcinoma, follicular thyroid carcinoma; medullary thyroid carcinoma, multiple endocrine neoplasia type 2A, multiple endocrine neoplasia type 2B, familial medullary thyroid cancer, pheochromocytoma, paraganglioma), pancreatic cancer (e.g., pancreatic andenocarcinoma, intraductal papillary mucinous neoplasm (IPMN), Islet cell tumors, ductal adenocarcinoma, insulinoma, glucagonoma, vipoma), adrenal gland cancer, neuroendocrine cancer (e.g., gastroenteropancreatic neuroendoctrine tumor (GEP-NET), carcinoid tumor), sebaceous gland carcinoma, sweat gland carcinoma). In certain embodiments, the cancer is sweat gland cancer (e.g., sweat gland carcinoma).

In certain embodiments, the cancer is head and neck cancer (e.g., squamous cell carcinoma of the head and neck (SCCHN), adenoid cystic carcinoma).

In certain embodiments, the cancer is oral cancer (e.g., buccal cavity cancer, lip cancer, tongue cancer, mouth cancer, pharynx cancer, hypopharynx cancer (e.g., hypopharyngeal carcinoma), throat cancer (e.g., laryngeal cancer, pharyngeal cancer, nasopharyngeal cancer, oropharyngeal cancer), salivary gland cancer).

In certain embodiments, the cancer is esophageal cancer (e.g., esophageal squamous cell carcinoma, esophageal adenocarcinoma, Barrett's adenocarcinoma, esophageal leiomyosarcoma).

In certain embodiments, the cancer is gastrointestinal cancer (e.g., anal cancer, colorectal cancer (e.g., colon cancer, rectal cancer, colorectal adenocarcinoma), gall bladder cancer, gastric cancer (e.g., stomach cancer (e.g., stomach adenocarcinoma)), gastrointestinal stromal tumor (GIST), small bowel cancer (e.g., appendix cancer, small bowel carcinoma, e.g., small bowel adenocarcinoma), small intestine cancer, large bowel cancer, large intestine cancer).

In certain embodiments, the cancer is cardiovascular cancer (e.g., primary cardiac tumors, angiosarcoma (e.g., lymphangiosarcoma, lymphangioendotheliosarcoma, hemangiosarcoma), endotheliosarcoma (e.g., Kaposi's sarcoma, multiple idiopathic hemorrhagic sarcoma), cardiac myxoma, cardiac rhabdomyoma).

In certain embodiments, the cancer is lung cancer (e.g., bronchus cancer (e.g., bronchogenic carcinoma, bronchial adenoma), alveolar carcinoma, mesothelioma, small cell lung cancer (SCLC), non-small cell lung cancer (NSCLC), lung adenocarcinoma, chondromatous hamartoma, papillary adenocarcinoma).

In certain embodiments, the cancer is a genitourinary cancer (e.g., bladder cancer (e.g., urothelial carcinoma), urethral cancer, kidney cancer (e.g., nephroblastoma a.k.a. Wilms' tumor, renal cell carcinoma), testicular cancer (e.g., seminoma, testicular embryonal carcinoma), germ cell cancer, prostate cancer (e.g., prostate adenocarcinoma), penile cancer (e.g., Paget's disease of the penis and scrotum)).

In certain embodiments, the cancer is a gynecological cancer (e.g., breast cancer (e.g., adenocarcinoma of the breast, papillary carcinoma of the breast, mammary cancer, medullary carcinoma of the breast, triple negative breast cancer, HER-2 positive breast cancer, HER2-negative breast cancer), endometrial cancer (e.g., uterine cancer (e.g., uterine sarcoma, choriocarcinoma), endometrial carcinoma), cervical cancer (e.g., cervical adenocarcinoma), ovarian cancer (e.g., cystadenocarcinoma, ovarian embryonal carcinoma, ovarian adenocarcinoma), germ cell cancer, vulvar cancer (e.g., Paget's disease of the vulva) vaginal cancer, fallopian tube cancer).

In certain embodiments, the cancer is skin cancer (e.g., squamous cell carcinoma (SCC), keratoacanthoma (KA), melanoma, basal cell carcinoma (BCC), dermatofribroma).

In certain embodiments, the cancer is a soft tissue cancer (e.g., intraepithelial neoplasms, epithelial carcinomas, epithelial sarcomas, adenocarcinomas, adenomas, fibrosarcomas, fibromas, liposarcomas, lipomas, myxomas, teratomas).

In certain embodiments, treating cancer can result in a reduction in size or volume of a tumor. For example, after treatment, tumor size is reduced by 5% or greater (e.g., 10%, 20%, 30%, 40%, 50%, 60%, 70%, 80%, 90% or greater) relative to its size prior to treatment. Size of a tumor may be measured by any reproducible means of measurement. The size of a tumor may be measured as a diameter of the tumor or by any reproducible means of measurement.

In certain embodiments, treating cancer can result in a reduction in the rate of growth or metastasis of the cancer or tumor. For example, after treatment, the rate of growth is reduced by 5% or greater (e.g., 10%, 20%, 30%, 40%, 50%, 60%, 70%, 80%, 90% or greater) relative to the rate of growth prior to treatment.

In certain embodiments, treating cancer may further result in a decrease in the number of tumors. For example, after treatment, tumor number is reduced by 5% or greater (e.g., 10%, 20%, 30%, 40%, 50%, 60%, 70%, 80%, 90% or greater) relative to number prior to treatment. Number of tumors may be measured by any reproducible means of measurement.

The number of tumors may be measured by counting tumors visible to the naked eye or at a specified magnification (e.g., 2×, 3×, 4×, 5×, 10×, or 50×).

In certain embodiments, treating cancer can result in a decrease in the number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to the number prior to treatment. The number of metastatic nodules may be measured by any reproducible means of measurement. The number of metastatic nodules may be measured by counting metastatic nodules visible to the naked eye or at a specified magnification (e.g., 2×, 10×, or 50×).

In certain embodiments, treating cancer can result in an increase in average survival time of a population of subjects treated according to the present disclosure in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the present disclosure. An increase in average survival time of a population may also be measured, for example, by calculating for a population the average length of survival following completion of a first round of treatment with the compound of the present disclosure.

In certain embodiments, treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with the compound of the present disclosure. A decrease in the mortality rate of a population may also be measured, for example, by calculating for a population the average number of disease-related deaths per unit time following completion of a first round of treatment with the compound of the present disclosure.

In certain embodiments, treating cancer can also result in an increased average progression-free survival time of a population of treated subjects in comparison to an untreated population. For example, the average progression-free survival time is increased by more than 30 days (more than 60 days, 90 days, or 120 days). An increase in average progression-free survival time of a population may be measured by any reproducible means. An increase in average progression-free survival time of a population may be measured, for example, by calculating for a population the average length of progression-free survival following initiation of treatment with the compound of the present disclosure. An increase in average progression-free survival time of a population may also be measured, for example, by calculating for a population the average length of progression-free survival following completion of a first round of treatment with the compound of the present disclosure. “Progression-free survival” as used herein refers to the length of time during and after medication or treatment during which the disease being treated (e.g., cancer) does not get worse.

In another aspect, provided herein are methods of stimulating macrophages to remove pathogenic cells by macroptosis in a subject in need thereof comprising administering to the subject an effective amount of an agent that stimulates macroptosis of pathogenic cells. In certain embodiments, the agent is IFN-ω, an analog thereof, or agent that increases expression of IFN-ω. In certain embodiments, the agent is a fusion protein described herein comprising IFN-ω or an analog thereof. In certain embodiments, the subject has a proliferative disease (e.g., cancer), infectious disease (e.g., chronic infection), autoimmune disease, neurodegenerative disease, atherosclerosis, metabolic disorder, or is in need of a conditioning regimen for stem cell transplant. Therefore also provided herein are methods, compositions, and uses for treating the aforementioned diseases and conditions.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. In certain embodiments, the subject is a human.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount effective for treating a disease (e.g., a proliferative disease such as cancer or a tumor) in a subject. In certain embodiments, a therapeutically effective amount is an amount effective for increasing macrophage-mediated cytotoxicity in a subject.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for preventing a disease (e.g., a proliferative disease) in a subject. In certain embodiments, a prophylactically effective amount is an amount effective for increasing macrophage-mediated cytotoxicity in a subject.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1: IFN-ω Activates Macrophages Against Colorectal Cancer

As shown in FIG. 1, IFN-alpha, -gamma, and -omega activate macrophages to inhibit DLD1 cells (without CD47 blockade).

Materials and methods. Primary human macrophages were co-cultured in 384-well plates with GFP+ DLD-1 cancer cells. Macrophages and cells were co-cultured in the presence of interferon alpha 2A (IFNA2A), interferon alpha 2B (IFNA2B), interferon omega (IFNW1), and interferon gamma (IFNG), all at a final concentration 1000 ng/ml, or PBS control. GFP+ area was quantified at 7 days by automated microscopy and image analysis (Incucyte®). Cell numbers per well: Human macrophages: 10,000 (4 donors, each in triplicate); DLD-1: 7500.

### Example 2: IFN-ω Activates Macrophages Against Breast Cancer Cells with Mesenchymal Properties

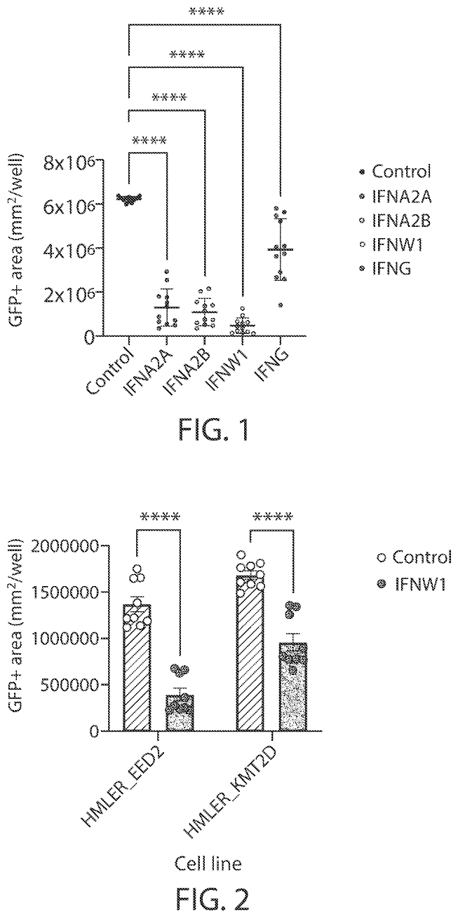

As shown in FIG. 2, IFN-ω can activate macrophages against cell lines with mesenchymal properties (without CD47 blockade).

Materials and methods. Primary human macrophages were co-cultured in 384-well plates with GFP+ HMLER cancer cells with knockout of the EED or KMT2D genes (described in Zhang, Y., Donaher, J. L., Das, S. et al., “Genome-wide CRISPR screen identifies PRC2 and KMT2D-COMPASS as regulators of distinct EMT trajectories that contribute differentially to metastasis.” Nat Cell Biol. 2022, 24, 554-564). Macrophages and cells were co-cultured in the presence of interferon omega (IFNW1) at a final concentration 1000 ng/ml, or PBS control. GFP+ area was quantified at 7 days by automated microscopy and image analysis (Incucyte®). Cell numbers per well: Human macrophages: 10,000 (3 donors, each in triplicate); HMLER: 7500.

### Example 3: Human IFN-ω Activates Murine Macrophages Against Colorectal Cancer

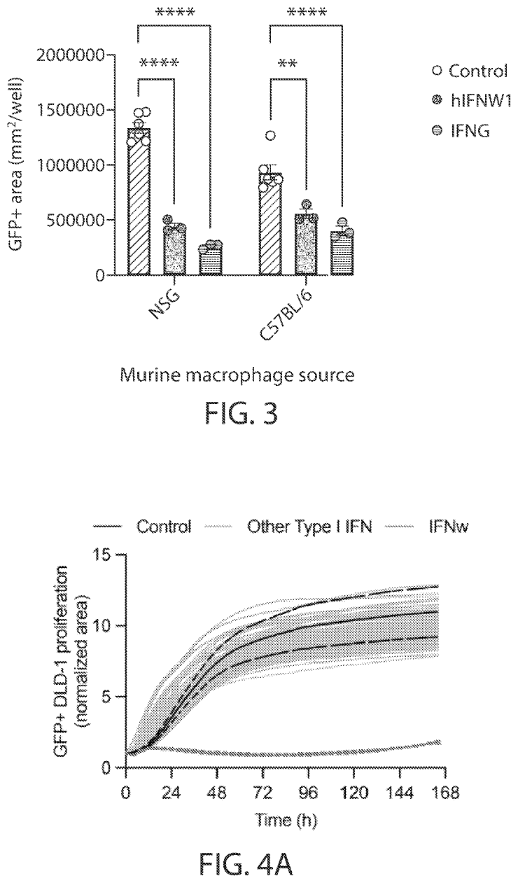

As shown in FIG. 3, human IFN-ω can activate murine macrophages against murine MC38 cells (without CD47 blockade).

Materials and methods. Murine C57BL/6 and NOD.Cg-Prkdc^{scid} Il2rg^{tm1Wjl}/SzJ (NSG) macrophages were co-cultured in 384-well plates with GFP+ MC38 cancer cells. Macrophages and cells were co-cultured in the presence of interferon omega (IFNW1), interferon gamma (IFNG) or PBS control. Cells were co-cultured for up to 7 days and GFP+ area was quantified by automated microscopy and image analysis (Incucyte®). Each condition was plated in triplicate. Cell numbers per well: Murine macrophages: 10,000; MC38: 1000.

### Example 4: IFN-ω Activates Macrophage-Dependent Cell Death of Cancer Cells

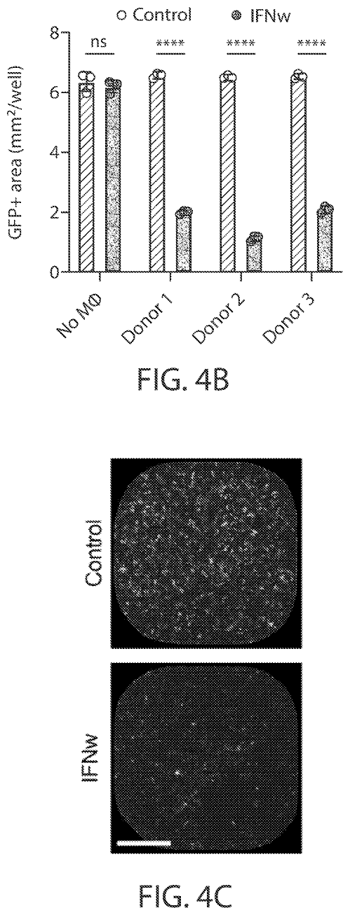

FIG. 4A demonstrates the results of a high-throughput screen performed using primary human macrophages that were co-cultured with GFP+ DLD-1 colon cancer cells. Cells were exposed to a library of recombinant human cytokines over the duration of co-culture. Each curve represents an individual cytokine. The black curve shows vehicle control samples with standard deviation (hashed lines). Cytokines that directly killed cancer cells in the absence of macrophages were excluded for clarity. FIG. 4B shows the validation of IFN-ω as a cytokine that specifically induced macrophage-dependent cell death using macrophages derived from additional human blood donors. ns, not significant, ***p<0.0001 by two-way ANOVA with correction for multiple comparisons. FIG. 4C shows the representative whole-well images of co-culture assays showing growth of GFP+ DLD-1 cells in the presence of primary human macrophages (unlabeled) following treatment with vehicle control versus recombinant human IFN-ω. Scale bar, 800 um.

### Example 5: IFN-ω Activates Macroptosis of Human and Mouse Cell Lines

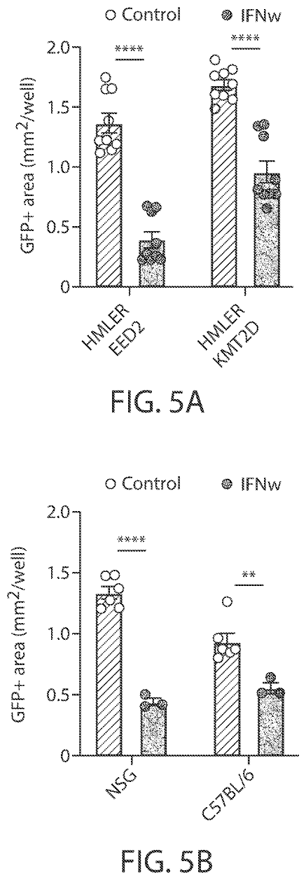

FIG. 5A demonstrates a co-culture of primary human macrophages with two variants of HMLER breast cancer cells which are enhanced for mesenchymal properties. FIG. 5B demonstrates a co-culture of primary mouse bone marrow-derived macrophages with MC38 mouse colorectal cancer cells. Macrophages deriving from immunocompromised mice (NSG) and wild-type mice (C57BL/6) both exerted macrophage-dependent cytotoxicity in response to human IFN-ω. FIGS. 5A-5B **p<0.01, ****p< by two-way ANOVA with correction for multiple comparisons.

### Example 6: Macroptosis is a Non-Phagocytic, Contact-Dependent Form of Macrophage-Induced Cell Death

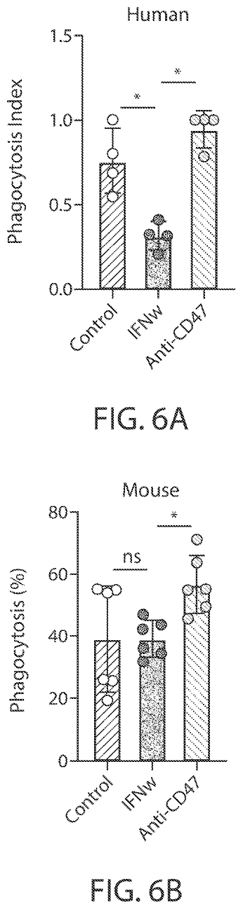

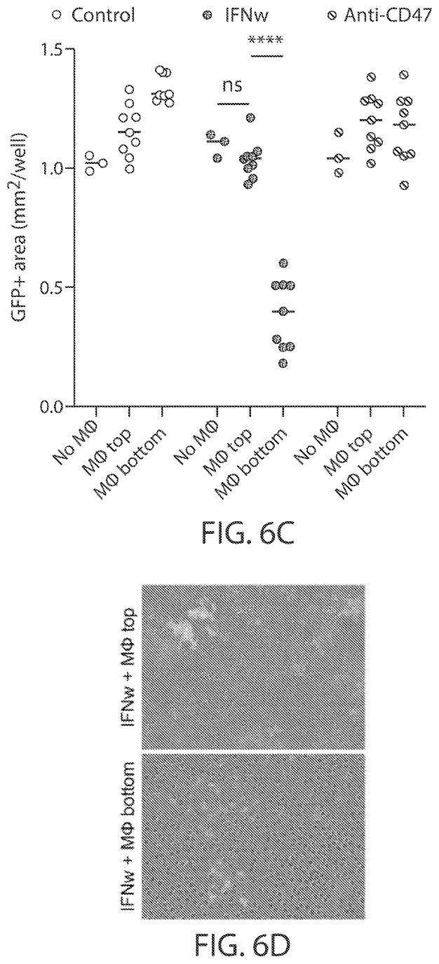

FIG. 6A demonstrates how phagocytosis assays were performed in which primary macrophages were exposed to control treatment or primed with 100 ng/mL human IFN-ω for 48 hours. Macrophages were harvested, then co-cultured with cancer cells for 2 hours. Phagocytosis was measured by flow cytometry as the percentage of macrophages engulfing GFP+ cancer cells. Phagocytosis assays were performed with human macrophages and DLD-1 cancer cells (FIG. 6A) or C57BL/6 mouse macrophages and MC38 cancer cells (FIG. 6B). In each case, no significant increase in phagocytosis was observed with IFN-ω. Anti-CD47 antibody was included as a positive control for phagocytosis. FIG. 6C exhibits a transwell assay demonstrating that macroptosis is a contact-dependent process. GFP+ DLD-1 cells were cultured in the bottom of a transwell chamber. Primary human macrophages were added to either the bottom or top chamber with shared medium. Cells were exposed to vehicle control, IFN-ω, or an anti-CD47 antibody. Macroptosis only occurred when macrophages were in direct contact with DLD-1 cells in the bottom chamber. The representative images in FIG. 6D were from transwell assays which showed how wells exposed to IFN-ω. When macrophages were seeded in the top chamber, a confluent monolayer of GFP+ DLD-1 cells formed on the bottom of the well. When macrophages were seeded on the bottom, macroptosis occurred and macrophages dispersed in the well with scattered GFP+ cells remaining. ns, ****p<0.0001 by one-way (FIGS. 6A-6B) or two-way ANOVA (FIG. 6C) with correction for multiple comparisons. The experiments were performed with n=3-4 independent macrophage donors.

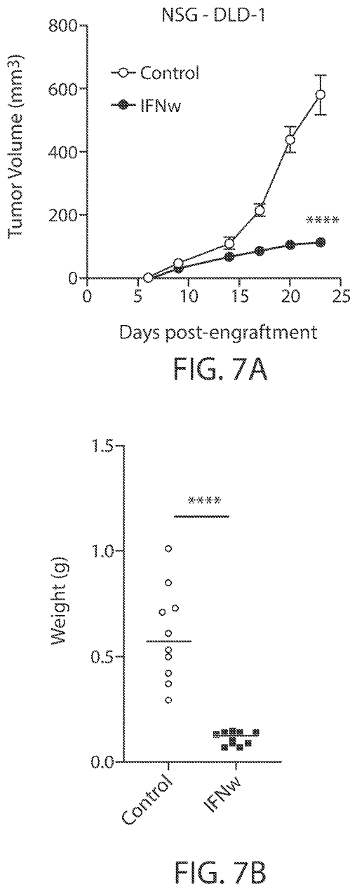

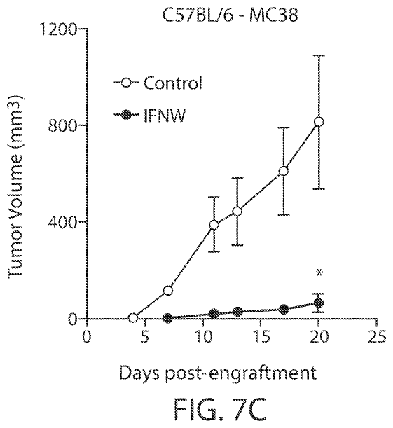

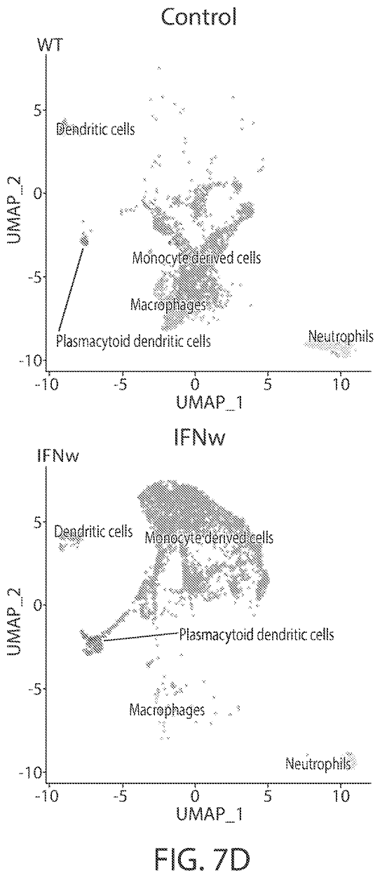

### Example 7: IFN-ω Stimulates Anti-Tumor Responses by Myeloid Cells In Vivo

in which Control DLD-1 cells were compared to a DLD-1 variant that secretes human IFN-ω, based off a xenograft model in NSG mice (FIG. 7A). N=10 mice per cohort. FIG. 7B demonstrates tumor weights at the end of the experiment from FIG. 7A. FIG. 7C shows an immunocompetent model in which wild-type C57BL/6 mice were engrafted with MC38 cells. A vector control cell line was compared to a cell line that secretes human IFN-ω. N=5 mice per cohort. FIG. 7D showed UMAP plots from scRNA-seq of sorted CD45+ cells from FIG. 7A, which showed that transcriptional changes predominantly occur in monocyte-lineage and macrophage populations, because of IFN-ω. FIGS. 7A-7C *p<0.05, ****p<0.0001 by two-tailed t-test.

### Example 8: CRISPR Screening Identifies the Genes that are Required for Sensitivity and Resistance to Macroptosis

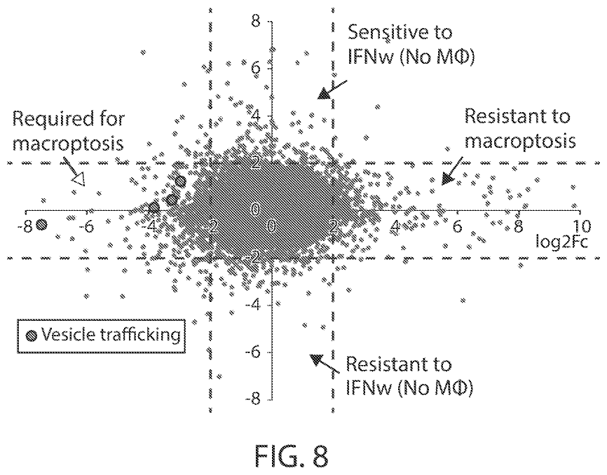

See FIG. 8. A pooled genomic screen was performed using a genome-wide CRISPRi sgRNA library that was introduced into DLD-1 cells. The cells were cultured alone, with IFN-ω, with human macrophages, or with human macrophages and IFN-ω for a period of 5 days. The surviving DLD-1 cells were then collected and sgRNAs were recovered by NGS. The summary results from the screen are presented as a scatter plot depicting log 2 fold-change (log 2FC) in effect size for IFN-ω treatment, versus control with DLD-1 cells alone (y-axis), or in the presence of macrophages to induce macroptosis (x-axis). An effect-size threshold of log 2FC ±2 (hashed lines) was used to define genes influence macrophage-dependent cell death and therefore are required for sensitivity or resistance to macroptosis. The highlighted genes were critical for vesicle trafficking.

### Example 9: IFN-ω-Fc Fusion Proteins Bind to Cancer Cells and Stimulate Macroptosis

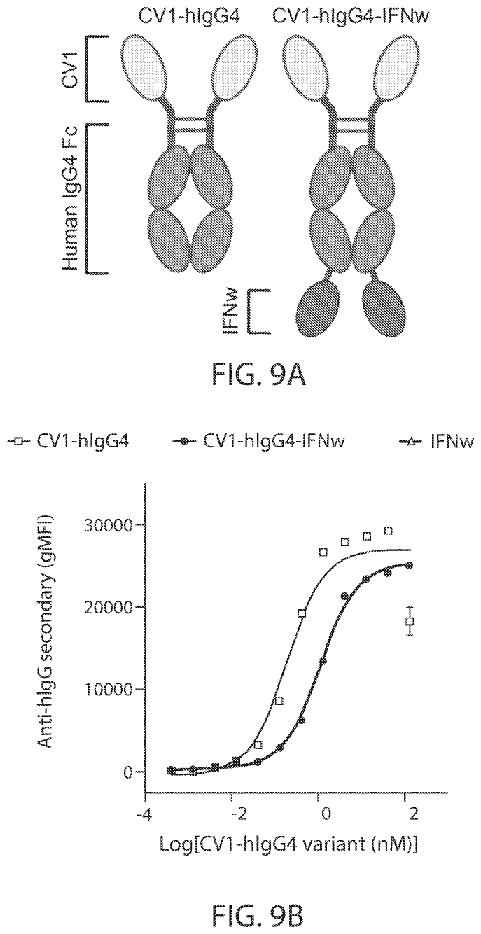

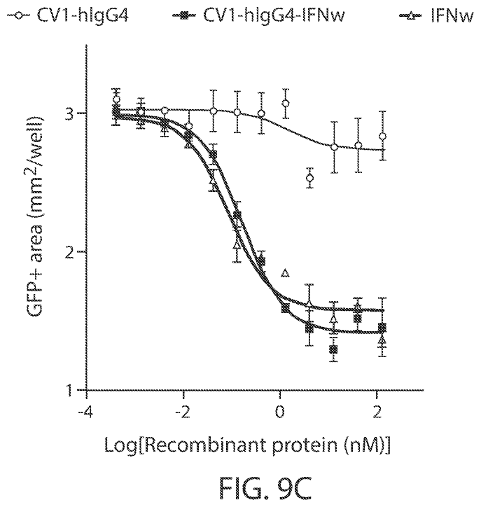

The initial design of Fc fusion proteins that was used as proof-of-concept is demonstrated by the schematic in FIG. 9A. CV1 was a high-affinity CD47-binding domain. The IFN-ω fusion protein contained a fusion of IFN-ω to the C-terminus of a human IgG4 Fc (S228P). FIG. 9B shows the binding of CV1-hIgG4 or an IFN-ω fusion protein to DLD-1 cells. Binding was detected by flow cytometry with an anti-human IgG secondary antibody. FIG. 9C shows a co-culture assay using primary human macrophages and GFP+ DLD-1 cells. The cells were cultured with titrating concentrations of CV1-hIgG4, CV1-hIgG4-IFN-ω, or IFN-ω. CV1-hIgG4-IFN-ω stimulated macroptosis, exerted a greater anti-tumor effect than CV1-hIgG4, and exhibited the same potency as IFN-ω. n=3 independent macrophage donors.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of treating a proliferative disease in a subject in need thereof comprising administering to the subject a therapeutically effective amount of interferon-omega (IFN-ω) or an analog thereof.

2. The method of claim 1, wherein the IFN-ω or analog thereof is administered in an amount effective to increase macrophage-mediated cytotoxicity in the subject, relative to control.

3. The method of claim 2, wherein the macrophage-mediated cytotoxicity is increased by at least 10%, relative to control.

4. The method of any one of claims 1-3, wherein the IFN-ω or analog thereof is human wild-type IFN-ω.

5. A method of treating a proliferative disease in a subject in need thereof comprising administering to the subject a therapeutically effective amount of an agent that increases endogenous interferon-omega (IFN-ω) expression in the subject, relative to control.

6. The method of claim 5, wherein the endogenous IFN-ω expression in the subject is increased by at least 10%, relative to control.

7. The method of claim 5 or 6, wherein the agent is administered in an amount effective to increase macrophage-mediated cytotoxicity in the subject, relative to control.

8. The method of claim 7, wherein the macrophage-mediated cytotoxicity is increased by at least 10%, relative to control.

9. The method of any one of claims 1-8, wherein the proliferative disease is cancer.

10. The method of claim 9, wherein the cancer is colorectal cancer.

11. The method of claim 10, wherein the colorectal cancer is colon adenocarcinoma.

12. The method of claim 9, wherein the cancer is breast cancer.

13. The method of any one of claims 1-12, wherein the subject has a tumor.

14. The method of claim 13, wherein the tumor has tumor-infiltrating macrophages (TAMs).

15. A method comprising administering to a subject interferon-omega (IFN-ω) or an analog thereof in an amount effective to increase macrophage-mediated cytotoxicity in the subject, relative to control.

16. The method of claim 15, wherein the IFN-ω or analog thereof is human wild-type IFN-ω.

17. A method comprising administering to a subject an agent that increases endogenous interferon-omega (IFN-ω) expression in the subject, relative to control, in an amount effective to increase macrophage-mediated cytotoxicity in the subject, relative to control.

18. The method of any one of claims 15-17, wherein the macrophage-mediated cytotoxicity is increased by at least 10%, relative to control.

19. The method of any one of claims 15-18, wherein the macrophages are tumor-associated macrophages (TAMs).

20. The method of any one of claims 15-19, wherein the subject is diagnosed with cancer.

21. The method of any one of claims 15-19, wherein the subject has a tumor.

22. The method of claim 21, wherein the tumor has tumor-infiltrating macrophages.

23. The method of any one of claims 1-22, wherein the subject is a human.

24. The method of any one of the preceding claims, wherein the agent that increases endogenous interferon-omega (IFN-ω) expression is a small molecule, protein or polypeptide, antibody or antigen-binding fragment thereof, or nucleic acid.

25. The method of claim 24, wherein the agent that increases endogenous interferon-omega (IFN-ω) expression is a nucleic acid that encodes IFN-ω or an analog thereof.

26. The method of claim 25, wherein the nucleic acid that encodes IFN-ω or an analog thereof is an mRNA.

27. The method of any one of the preceding claims, wherein the IFN-ω, analog thereof, or agent that increases endogenous IFN-ω expression is conjugated to an anti-cancer antibody for targeted delivery to a tumor or cancer.

28. The method of claim 27, wherein the anti-cancer antibody is rituximab, cetuximab, or trastuzumab.

29. A composition for use in treating a proliferative disease comprising IFN-ω, an analog thereof, or an agent that increases endogenous IFN-ω expression.

30. Use of IFN-ω, an analog thereof, or an agent that increases endogenous IFN-ω expression, for the preparation of a medicament for treating a proliferative disease.

31. A method of stimulating macrophages to remove pathogenic cells by macroptosis in a subject in need thereof comprising administering to the subject an effective amount of an agent that stimulates macroptosis of pathogenic cells.

32. The method of claim 31, wherein the removal of pathogenic cells by macroptosis is increased by at least 10%, relative to control.

33. The method of claim 31 or 32, wherein the agent is IFN-ω, an analog thereof, or agent that increases expression of IFN-ω.

34. The method of claim 31 or 32, wherein the agent is a fusion protein comprising IFN-ω or an analog thereof.

35. The method of any one of claims 31-34, wherein the subject has a proliferative disease, infectious disease, autoimmune disease, neurodegenerative disease, atherosclerosis, metabolic disorder, or is in need of a conditioning regimen for stem cell transplant.

36. A fusion protein comprising IFN-ω, or an analog thereof, and an antigen-binding domain.

37. The fusion protein of claim 36 comprising IFN-ω or an analog thereof, an Fc domain, and an antigen-binding domain.

38. The fusion protein of claim 37, wherein the Fc domain is a human IgG4 Fc domain.

39. The fusion protein of any one of claims 36-38, wherein the antigen-binding domain is a CD47-binding domain.

40. The fusion protein of claim 39, wherein the CD47-binding domain is CV1 or an analog thereof.

41. The fusion protein of any one of claims 37-40, wherein the antigen-binding domain is operably linked to the Fc domain, and the Fc domain is operably linked to the IFN-ω or analog thereof.

42. The fusion protein of claim 41, wherein the IFN-ω or analog thereof is operably linked to the C-terminus of the Fc domain.

43. The fusion protein of claim 42, wherein the IFN-ω or analog thereof is operably linked to the C-terminus of the Fc domain, and the Fc domain is operably linked to the C-terminus of the antigen-binding domain.

44. A method of treating a proliferative disease, infectious disease, autoimmune disease, neurodegenerative disease, atherosclerosis, or metabolic disorder in a subject in need thereof comprising administering to the subject a therapeutically effective amount of the fusion protein of any one of claims 36-43.

45. The method of claim 44 for treating a proliferative disease, wherein the proliferative disease is cancer.

46. The method of claim 45, wherein the cancer is colorectal cancer.

47. The method of claim 45 or 46, wherein the cancer is colon adenocarcinoma.

48. The fusion protein of any one of claims 36-44, for use in treating a proliferative disease, infectious disease, autoimmune disease, neurodegenerative disease, atherosclerosis, or metabolic disorder.

49. Use of the fusion protein of any one of claims 36-44, for use in the preparation of a medicament.

50. A method comprising:
(i) co-culturing cancer cells and macrophages with IFN-ω, an analog thereof, or an agent that increases expression of IFN-ω in the co-culture; and
(ii) detecting or measuring the number of cancer cells or rate of cancer cell proliferation.

51. The method of claim 50, wherein the cancer cells are obtained from a subject.

52. The method of claim 51 further comprising:
(iii) selecting the IFN-ω, analog thereof, or agent that increases expression of IFN-ω in the co-culture as a candidate for administration to the subject.

53. A method of identifying an agent that induces cancer cell death by macroptosis over phagocytosis, the method comprising:
(i) co-culturing cancer cells and macrophages with a candidate agent, wherein the cancer cells and macrophages are capable of direct contact with each other;
(ii) detecting or measuring the total level or rate of cancer cell death;
(iii) detecting or measuring the level or rate of cancer cell death due to phagocytosis; and
(iv) selecting the candidate agent as an agent that induces cancer cell death by macroptosis over phagocytosis if the total level or rate of cancer cell death is increased by a statistically significant amount, relative to control, and the level or rate of cancer cell death due to phagocytosis is not increased by a statistically significant amount, relative to control.

54. The method of claim 53, wherein the macrophages are primary human macrophages or C57BL/6 mouse macrophages.

55. The method of claim 53 or 54, wherein the cancer cells comprise GFP+ cancer cells.

56. The method of claim 55, wherein the level or rate of cancer cell death due to phagocytosis is measured by flow cytometry.

57. The method of any one of claims 53-56, wherein the cancer cells are obtained from a subject.

58. The method of any one of claims 53-57, wherein the cancer cells are DLD-1 cells or MC38 cells cancer cells.
